(19) United States
(12) Patent Application Publication
(10) Pub. No.: US 2026/0215702 A1
(43) Pub. Date: Jul. 30, 2026

(54) METHOD AND APPARATUS FOR DETERMINING CARDIAC MOTION FEATURE

(71) Applicant: Fujitsu Limited, Kawasaki-shi (JP)

(72) Inventors: Qian ZHAO, Beijing (CN); Hongchun LI, Beijing (CN); Jun TIAN, Beijing (CN)

(73) Assignee: Fujitsu Limited, Kawasaki-shi (JP)

(21) Appl. No.: 19/446,562

(22) Filed: Jan. 12, 2026

(30) Foreign Application Priority Data
Jan. 20, 2025 (CN) 202510088388.3

(51) Int. Cl. A61B 5/11; A61B 5/00; A61B 5/0507

(52) U.S. Cl. CPC A61B 5/1126; A61B 5/0507; A61B 5/7221; A61B 5/7225; A61B 5/725; A61B 5/7257; A61B 5/7264; A61B 5/7271

(57) ABSTRACT

An apparatus to determine a cardiac motion feature and a method thereof. The method includes: acquiring wireless signals reflected by multiple body parts via a wireless sensing device; filtering the acquired wireless signals using a single-band filter or a multi-band filter, to obtain filtered time domain signals; calculating signal quality evaluation values for evaluating signal quality of the filtered time domain signals; selecting candidate signals from the filtered time domain signals according to the signal quality evaluation values; and determining a cardiac motion feature based on the candidate signals. The method is easy to implement and easy to operate, which can not only guarantee accuracy of the obtained cardiac motion feature, but also has a strong anti-noise capability and high privacy protectiveness.

### CROSS-REFERENCE TO RELATED APPLICATIONS

This application is based on and hereby claims priority to Chinese Application No. 202510088388.3, filed Jan. 20, 2025, in the China National Intellectual Property Administration, the disclosure of which is incorporated herein by reference.

### TECHNICAL FIELD

The present disclosure relates to the field of physiological signal detection and analysis, in particular to a method and an apparatus for determining a cardiac motion feature.

### BACKGROUND ART

Heart is an vital organ of a human body, continuously monitoring and analyzing activities of the heart of the human body enables better diagnosing, controlling and preventing cardiovascular diseases, and promoting people to perform physical health management better. At present, cardiac motion monitoring is widely applied in fields such as patient monitoring systems, smart homes and smart elderly care, to provide health monitoring service for relevant groups (such as patients and the elderly). By monitoring an index value of cardiac motion such as heart rate variability, an emotional state of the human body such as anxiety, sadness, happiness, etc., may be judged, which may be used in the fields of emotion detection, lie detection, psychological analysis, etc. Therefore, it is of great social and economic significance to improve accuracy and convenience of cardiac motion detection.

Common cardiac motion monitoring methods include wearable device-based e.g. electrocardiogram and non-contact radar detection technologies, among which, a wearable device requires a user to wear the device for a long time, which limits reliability, adaptability and continuity of monitoring, and leads to poorer user experience. A radar-based physiological signal detection method has a wide range of application scenarios and good market competitiveness. Radar is a non-contact detection technology, a user does not need to wear a device on his/her body, which provides higher convenience and privacy.

It should be noted that the above introduction to the technical background is just to facilitate a clear and complete description of the technical solutions of the present disclosure, and is elaborated to facilitate understanding of persons skilled in the art. It cannot be considered that these technical solutions are known by persons skilled in the art just because these solutions are elaborated in the Background of the present disclosure.

### SUMMARY

According to an aspect of the embodiments of the present disclosure, an apparatus to determine a cardiac motion feature is provided. The apparatus comprises: a memory; and a processor coupled to the memory to: acquire wireless signals reflected by multiple body parts via a wireless sensing device; filter the acquired wireless signals using a single-band filter or a multi-band filter, to obtain filtered time domain signals; calculate signal quality evaluation values for evaluating signal quality of the filtered time domain signals; select candidate signals from the filtered time domain signals according to the signal quality evaluation values; and determine a cardiac motion feature based on the candidate signals.

According to another aspect of the embodiments of the present disclosure, a method to determine a cardiac motion feature is provided, the method including: acquiring wireless signals reflected by multiple body parts via a wireless sensing device; filtering the acquired wireless signals using a single-band filter or a multi-band filter, to obtain filtered time domain signals; calculating signal quality evaluation values for evaluating signal quality of the filtered time domain signals; selecting candidate signals from the filtered time domain signals according to the signal quality evaluation values; and determining a cardiac motion feature based on the candidate signals.

According to a further aspect of the embodiments of the present disclosure, a computer equipment is provided, the computer equipment comprising a memory and a processor, the memory storing a computer program, the processor being configured to execute the computer program to carry out the method as described above.

According to another aspect of the embodiments of the present disclosure, a storage medium storing a computer readable program is provided, the computer readable program causing a computer to execute the method as described above.

One of advantageous effects of the embodiments of the present disclosure lies in: according to the embodiments of the present disclosure, a cardiac motion feature is determined in a non-contact manner, by performing processing such as filtering, quality evaluation and selection on obtained wireless signals, accuracy of the obtained cardiac motion feature is ensured, and it is also easy to implement, simple to operate, and has a strong anti-noise capability and high privacy protectiveness.

Referring to the later description and drawings, specific implementations of the present disclosure are disclosed in detail, indicating a mode that the principle of the present disclosure may be adopted. It should be understood that the implementations of the present disclosure are not limited in terms of a scope. Within the scope of the terms of the attached claims, the implementations of the present disclosure include many changes, modifications and equivalents.

Features that are described and/or shown for one implementation may be used in the same way or in a similar way in one or more other implementations, may be combined with or replace features in the other implementations.

It should be emphasized that the term “comprise/include” when being used herein refers to presence of a feature, a whole piece, a step or a component, but does not exclude presence or addition of one or more other features, whole pieces, steps or components.

### BRIEF DESCRIPTION OF DRAWINGS

An element and a feature described in a drawing or an implementation of the embodiments of the present disclosure may be combined with an element and a feature shown in one or more other drawings or implementations. In addition, in the drawings, similar labels represent corresponding components in several drawings and may be used to indicate corresponding components used in more than one implementation.

The included drawings are used to provide a further understanding on the embodiments of the present disclosure, constitute a part of the Specification, are used to illustrate the implementations of the present disclosure, and expound the principle of the present disclosure together with the text description. Obviously, the drawings in the following description are only some embodiments of the present disclosure. Persons skilled in the art may further obtain other drawings according to these drawings under the premise that they do not pay inventive labor. In the drawings:

FIG. 1 is a schematic diagram of a method for determining a cardiac motion feature according to an embodiment of the present disclosure;

FIG. 2 is a schematic diagram of operation in the method shown in FIG. 1 according to an embodiment of the present disclosure;

FIG. 3 is another schematic diagram of a method for determining a cardiac motion feature according to an embodiment of the present disclosure;

FIG. 4 is a schematic diagram of operation in the method shown in FIG. 1 according to an embodiment of the present disclosure;

FIG. 5 is a further schematic diagram of a method for determining a cardiac motion feature according to an embodiment of the present disclosure;

FIG. 6 is a schematic diagram of an implementation scenario according to an embodiment of the present disclosure;

FIG. 7 is a curve graph of an evaluation result according to an embodiment of the present disclosure;

FIG. 8A is a curve graph of an experiment result according to an embodiment of the present disclosure;

FIG. 8B is another curve graph of an experiment result according to an embodiment of the present disclosure;

FIG. 9 is a schematic diagram of an apparatus for determining a cardiac motion feature according to an embodiment of the present disclosure;

FIG. 10 is a schematic diagram of a computer equipment according to an embodiment of the present disclosure.

### DETAILED DESCRIPTION

Referring to the drawings, through the following Specification, the aforementioned and other features of the present disclosure will become obvious. The Specification and the drawings specifically disclose particular implementations of the present disclosure, showing partial implementations which may adopt the principle of the present disclosure. It should be understood that the present disclosure is not limited to the described implementations, on the contrary, the present disclosure includes all the modifications, variations and equivalents falling within the scope of the attached claims.

In the embodiments of the present disclosure, the term “first” and “second”, etc. are used to distinguish different elements in terms of appellation, but do not represent a spatial arrangement or time sequence, etc. of these elements, and these elements should not be limited by these terms. The term “and/or” includes any and all combinations of one or more of the associated listed terms. The terms “include”, “comprise” and “have”, etc. refer to the presence of stated features, elements, members or components, but do not preclude the presence or addition of one or more other features, elements, members or components.

In the embodiments of the present disclosure, the singular forms “a/an” and “the”, etc. include plural forms, and should be understood broadly as “a kind of” or “a type of”, but are not defined as the meaning of “one”; in addition, the term “the” should be understood to include both the singular forms and the plural forms, unless the context clearly indicates otherwise. In addition, the term “according to” should be understood as “at least partially according to . . . ”, the term “based on” should be understood as “at least partially based on . . . ”, unless the context clearly indicates otherwise.

The inventor finds that the existing non-contact cardiac signal detection methods mainly focus on monitoring a heart rate, and mostly use methods such as single-band filtering or signal decomposition to perform signal analysis, accuracy and convenience of detection need to be improved.

For at least one of the above problems or other similar problems, embodiments of the present disclosure provide a method and an apparatus for determining a cardiac motion feature based on multi-frequency band and multi-location, which may obtain cardiac motion signals of a human body and thereby determine a cardiac motion feature by performing single-band and multi-band filtering on radar reflected signals of multiple body parts, performing signal quality evaluation on a filtered signal and selecting signals with higher signal quality.

In the embodiments of the present disclosure, radar may be a SFCW (Stepped Frequency Continuous Waveform) radar or a FMCW (Frequency Modulated Continuous Wave) radar, but is not limited to this. The radar transmits electromagnetic waves via a transmitting antenna, and receives corresponding reflected waves (which may be called radar echo information or a radar echo signal) after being reflected by different objects. By processing and analyzing the radar echo information, information such as a physiological signal of a detection target can be extracted, such information can meet requirements of many application scenarios.

In the embodiments of the present disclosure, the detection target may be persons of various ages, such as the elderly, children, the elderly and/or nursing staff, children and/or custodians. The present disclosure is not limited to this, the detection target may further be an animal with a life feature, and so on. The human body is taken as an example for the following description.

Various implementations of the embodiments of the present disclosure will be described below with reference to the drawings.

### Embodiments of a First Aspect

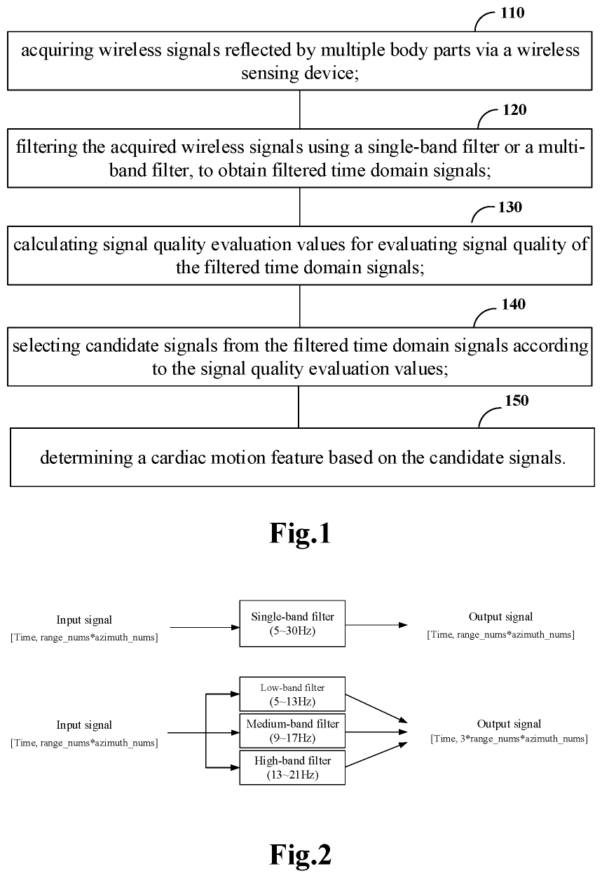

Embodiments of the present disclosure provide a method for determining a cardiac motion feature. FIG. 1 is a schematic diagram of a method for determining a cardiac motion feature in the embodiments of the present disclosure. As shown in FIG. 1, the method includes:
110, acquiring wireless signals reflected by multiple body parts via a wireless sensing device;
120, filtering the acquired wireless signals using a single-band filter or a multi-band filter, to obtain filtered time domain signals;
130, calculating signal quality evaluation values for evaluating signal quality of the filtered time domain signals;
140, selecting candidate signals from the filtered time domain signals according to the signal quality evaluation values; and
150, determining a cardiac motion feature based on the candidate signals.

It should be noted that the above FIG. 1 only schematically describes the embodiments of the present disclosure, but the present disclosure is not limited to this. For example, an execution step of each operation may be adjusted appropriately, moreover other some operations may be increased or reduced. Persons skilled in the art may make appropriate modifications according to the above contents, not limited to the records in the above FIG. 1.

According to the above embodiments, for radar reflected signals of multiple body parts, performing single-band or multi-band filtering, performing signal quality evaluation on a filtered signal, selecting candidate signals based on signal quality evaluation values, and determining a cardiac motion feature based on the candidate signals, thereby, the cardiac motion feature is determined in a non-contact manner, accuracy of the obtained cardiac motion feature is ensured, and it is also easy to implement, simple to operate, and has a strong anti-noise capability and high privacy protectiveness.

In the above embodiments, in the operation 110, a wireless device such as a SFCW Stepped Frequency Continuous Waveform (SFCW) radar or a Frequency Modulated Continuous Wave (FMCW) radar may be used to obtain reflected signals of different body parts of a human body, for example, to obtain an I/Q signal of different body parts of the human body or an amplitude of the signal or a phase of the signal. For specific contents of signal acquisition, relevant technologies in this field may be referred to, and they are not described here.

In the operation 120, the above obtained wireless signals is taken as an input signal, on which single-band filtering or multi-band filtering is performed, to obtain a filtered time domain signal. Since the wireless signal obtained in the operation 110 is a reflected signal of different body parts of the human body, in order to further select a signal near to a human heart area, a filtering operation needs to be performed on the reflected signal (i.e., the input signal).

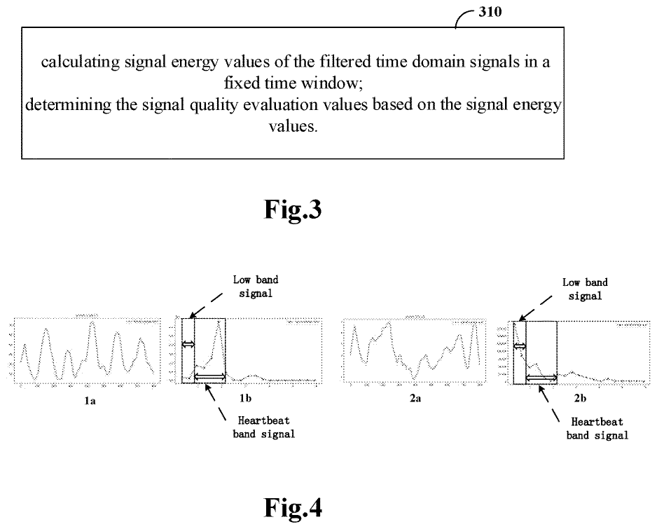

FIG. 2 is a schematic diagram of operation 120 in the method shown in FIG. 1. In general, a heartbeat frequency of the human body is 8 to 24 Hz. Therefore, as shown in FIG. 2, the input signal is respectively denoted as [Time, range_nums*azimuth_nums], where “Time” refers to a moment of a signal, “range” refers to a distance between a human body and a radar, which can indicate a human body position denoted by the signal, “azimuth” refers to a position of a body corresponding to the signal relative to a horizontal direction of a wireless device, which can indicate a body part denoted by the signal. In the case of single-band filtering, a filter with a frequency of 5 to 30 Hz can cover range intervals of heartbeat frequencies, thereby obtaining a human heart position and nearby reflected signals. In the case of multi-band filtering, low-band, medium-band and high-band filters with frequencies of 5 to 13 Hz, 9 to 17 Hz and 13 to 21 Hz respectively are used to filter the obtained wireless signal.

The filtered time domain signals obtained via the operation 120 may be denoted as [Time, m*range_nums*azimuth_nums], where “m” denotes the number of bands. In the above embodiments, “m=1” indicates that a single-band filter is used, and “m=3” indicates that three bands (low band, medium band, and high band) are used for filtering. Therefore, as shown in FIG. 2, an output signal result obtained via the single-band filter is [Time, range_nums*azimuth_nums], and an output signal result obtained via three filters of low-band, medium-band and high-band is [Time, 3*range_nums*azimuth_nums].

Since a heartbeat frequency varies greatly among individuals, it can be applied to a wider group of people through division of multiple bands, and filter out signals with higher quality.

In the above embodiments, division of the three bands of low, medium and high in the multi-band filter and setting of each frequency interval are only a preferred example, different frequencies and different numbers of bands may further be set according to an actual application, the present disclosure does not make restrictions in this regard.

Via the operating 120, a time domain signal that is reflected at a position closest to a human heart is obtained in a case where there is no need to define an apparatus position of the wireless device and a user does not need to wear any wearable device, thereby there is high applicability while the quality of an acquired signal can be maintained.

In the above embodiments, in the operation 130, signal quality evaluation values of the signal quality of the filtered time domain signals are calculated, for example, the signal quality evaluation values are calculated according to a phase, an amplitude or a power spectral density diagram of the filtered time domain signal. A calculation method for the signal quality evaluation values will be described later.

After the signal quality evaluation values are obtained according to the above calculation, in the operation 140, a signal may be selected based on the signal quality evaluation values.

In some embodiments, in the operation 140, the signal quality evaluation values may be sorted, n filtered signals with higher signal quality evaluation values may be selected as candidate signals, where n is a natural number. For example, signal quality evaluation values are arranged in descending order, and the filtered signals whose index values are arranged in the first n ranks are selected as candidate signals.

In some other embodiments, in the operation 140, a signal quality evaluation threshold may be calculated based on a maximum value in the signal quality evaluation values, and filtered signals with signal quality evaluation values greater than the signal quality evaluation threshold may be selected as candidate signals. For example, the maximum value Scoreₘₐₓ in an obtained signal quality evaluation value sequence is calculated, and the signal quality evaluation threshold Scoreₜₕ is calculated based on the maximum value, and the filtered signals with signal quality evaluation values greater than the signal evaluation threshold are selected as candidate signals. For example, the signal quality evaluation threshold may be calculated according to the following formula (1):

$${Score}_{th} = α S c o r {e}_{max} ( 0.75 \leq α < 1 ) \qquad ( 1 )$$

Through the processing in operations 130 and 140, candidate signals with higher quality are selected from the filtered signals, and in the operation 150, a cardiac motion feature may be further determined based on the candidate signals.

In the above embodiments, a signal with better quality can be selected by evaluating and screening the quality of the filtered signals, thereby ensuring accuracy of the cardiac motion feature obtained based on the candidate signals.

Through the above embodiments, a cardiac motion feature is determined in a non-contact manner, by performing processing such as filtering, quality evaluation and selection on obtained wireless signals, accuracy of the obtained cardiac motion feature is ensured, and it is also easy to implement, simple to operate, and has a strong anti-noise capability and high privacy protectiveness.

FIG. 3 is another schematic diagram of a method for determining a cardiac motion feature in the embodiments of the present disclosure. As shown in FIG. 3, a method for determining a cardiac motion feature provided by the embodiments of the present disclosure further includes:
310, calculating signal energy values of the filtered time domain signals in a fixed time window.

In the above embodiment, in the operation 130, the signal quality evaluation values may be calculated based on the signal energy values.

For example, it is assumed that a signal amplitude of the filtered time domain signals obtained after the operation 120 is Sₐ, and a fixed time window in a time domain is Δt_{w}, thereby a convolution result Sₑ of signal energy of the filtered time domain signals in the time window Δt_{w} can be calculated, and the convolution result Sₑ is a representation of the signal energy values.

In the above embodiment, in the operation 130, in each fixed time window Δt_{w}, a signal energy value at each moment of the filtered time domain signals is equal to a sum of signal energy values at a current moment and preceding moments. For example, the signal energy value at the moment t may be denoted as S_{e_t}, as shown in the following formula (2).

$${S}_{e\_t} = {\sum }_{i = t - Δ {t}_{w} - 1}^{i = t} {S}_{a\_i}^{2} , 0.2 s \leq ( Δ {t}_{w} ) \leq 1 s \qquad ( 2 )$$

In the embodiments of the present disclosure, the above calculation is performed for the signal energy value at each moment of the filtered time domain signals, thereby, the signal energy value Sₑ of the filtered time domain signals can be obtained.

In the above embodiments, the length of the fixed time window Δt_{w} is a preset value. For example, in the formula (2), the range of Δt_{w} is 0.2 s to 1 s, this range is only an example, the present disclosure does not make restrictions in this regard. In order to obtain a better result, the filtered time domain signals in the fixed time window Δt_{w} includes one heartbeat cycle and is less than one respiratory cycle, thus being able to avoid noise interference from breathing on a signal.

In some embodiments, the signal quality evaluation values may further be calculated according to the above signal energy values. For example, in the operation 130, a fast Fourier transform is performed on signal energy values of the filtered time domain signals in the fixed time window Δt_{w}, i.e., time domain energy values Sₑ, to obtain frequency domain energy values of frequency domain signals, and the frequency domain signals are divided into low band signals and heartbeat band signals according to the frequency domain energy values of the frequency domain signals; and signal quality evaluation values are determined according to the low band signals and the heartbeat band signals.

FIG. 4 is a schematic diagram of operation 130 in the method shown in FIG. 1. As shown in FIG. 4, 1a and 2a denote time domain energy values of signals, and 1b and 2b denote corresponding frequency domain energy values, respectively. As shown by 1b and 2b in FIG. 4, the frequency domain signals are divided into low band signals (0.01 Hz<f<0.8 Hz) and heartbeat band signals (0.8 Hz≤f<2.3 Hz) according to the frequency domain energy values of the frequency domain signals.

In the above embodiment, according to the divided low band signals and heartbeat band signals, whether there is an extreme value point in a heartbeat band signal interval is further determined: when there is and only one extreme value point in a frequency domain energy value sequence of the heartbeat band, a ratio of a frequency domain energy value at the extreme value point to a maximum frequency domain energy value in the low band signals is calculated, which is taken as a signal quality evaluation value; when the number of extreme value points in the frequency domain energy value sequence of the heartbeat band is greater than 1, a maximum frequency domain energy value in all the extreme value points is calculated, then a ratio of the maximum frequency domain energy value to the maximum frequency domain energy value in the low band signals is calculated, which is taken as a signal quality evaluation value; and when there is no extreme value point in the frequency domain energy value sequence of the heartbeat band, a minimum frequency domain energy value in the heartbeat band signals is calculated, then a ratio of the minimum frequency domain energy value to the maximum frequency domain energy value in the low band signals is calculated, which is taken as a signal quality evaluation value.

As shown in FIG. 4, according to the above calculation method for the signal quality evaluation values, the signal quality evaluation value calculated based on signals 1a and 1b is 18.72, and the signal quality evaluation value calculated based on signals 2a and 2b is 0.31. Therefore, the signals represented by signals 1a and 1b are signals with better quality.

After the signal quality evaluation values are calculated according to the above method, in the operation 140, selection of candidate signals may be performed according to the method described above to obtain the candidate signals.

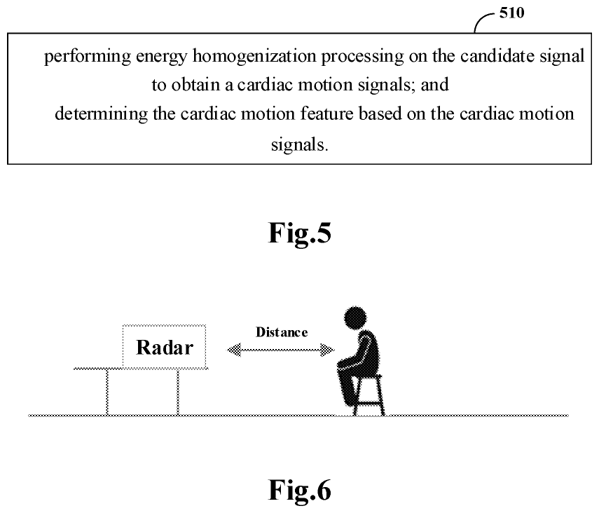

FIG. 5 is a further schematic diagram of a method for determining a cardiac motion feature in the embodiments of the present disclosure. As shown in FIG. 5, a method for determining a cardiac motion feature provided by the embodiments of the present disclosure further includes:

operation 510, performing energy homogenization processing on the candidate signals to obtain cardiac motion signals.

In some embodiments, in the operation 150, the cardiac motion feature may be determined based on the cardiac motion signals.

In some embodiments, in the operation 150, energy homogenization processing may be performed on the candidate signals to obtain candidate signals which has been subjected to energy homogenization processing. For example, k candidate signals obtained according to the method described above are denoted as [S_{a_1}, S_{a_2}, . . . , S_{a_k}], where Sₐ denotes an amplitude of a signal. After energy homogenization processing is performed on the k candidate signals along a time dimension, k candidate signals [s_{e_1_equal}, s_{e_2_equal}, . . . , s_{e_k_equal}] which have been subjected to energy homogenization processing can be obtained.

In the above embodiment, each candidate signal is a sequence of a time duration. For example, the k^{th} candidate signals S_{e_k_equal} which have been subjected to homogenization processing, is as shown in the following formula (3), contains values of moments such as t1, t2, . . . , t:

$${s}_{e\_k \_equal} = [ {s}_{e\_k \_t 1 \_equal} , {s}_{e\_k \_t 2 \_equal} , … , {s}_{e\_k \_t \_equal} ] \qquad ( 3 )$$

For each candidate signal which has been subjected to energy homogenization processing, for example, the k^{th} candidate signal, its value at moment t being equal to a ratio of an energy value at moment t to an energy at adjacent moments, which is shown in the following formulas (4) to (6):

$${s}_{e\_k \_t \_equal} = \frac{{s}_{e\_k \_t}}{{s}_{e\_k \_t \_neighbor}} \qquad ( 4 )$$

$${s}_{e\_k \_t} = {( {s}_{e\_k \_t} )}^{2} \qquad ( 5 )$$

$${s}_{e\_k \_t \_neighbor} = {\sum }_{i = t - Δ t}^{i = t + Δ t} {( {s}_{a\_k \_i} )}^{2} , Δ t = 0.5 s \qquad ( 6 )$$

In the above formula (6), k in S_{a_k_i} denotes k signals, and i denotes a moment.

The cardiac motion signals may be obtained after weighted summing or weighted averaging is performed on the candidate signals which have been subjected to energy homogenization processing according to the above formulas.

For example, weighted averaging may be performed on the candidate signals which have been subjected to energy homogenization processing in a manner shown in the following formula (7):

$${S}_{c a r d i a c} = ( {β}_{1} {S}_{e\_ 1 \_equal} + {β}_{2} {S}_{e \_ 2 \_ equal} + … + {β}_{k} {S}_{e\_k \_equal} ) / k \qquad ( 7 )$$

For another example, weighted summing may be performed on the candidate signals which have been subjected to energy homogenization processing in a manner shown in the following formula (8):

$${S}_{c a r d i a c} = ( {β}_{1} {S}_{e\_ 1 \_equal} + {β}_{2} {S}_{e \_ 2 \_ equal} + … + {β}_{k} {S}_{e\_k \_equal} ) \qquad ( 8 )$$

In the above formulas (7) and (8), a range of weight β is, for example, 0<β₁, β₂, . . . , βₖ≤1, which may be a pre-set value or may further be characterized by using a signal quality evaluation value. For example, a signal with better signal quality is set with a greater weight value, which has a greater contribution to acquisition of the cardiac motion signals.

According to the processing in the above operation 510, energies of signals at the same moment in different positions and signals at different moments in the same position are uniform, thereby influence of abnormal signals can be removed, and the accuracy of obtained cardiac motion signals can be improved.

According to the cardiac motion signals obtained by the above method, a cardiac motion feature can be further determined, such as a heartbeat rate, an IBI (Inter-Beat Interval), a HRV (Heart Rate Variability), etc.

Effects of the embodiments of the present disclosure are described below in conjunction with a specific example.

FIG. 6 is a schematic diagram of an implementation scenario in the embodiments of the present disclosure. As shown in FIG. 6, under this scenario, signals reflected at a heart position of a human body and signals reflected at a central position of the torso at different distances between a radar and the human body are collected, meanwhile, a wearable device is further used to collect cardiac motion features as data comparison. Collected data is shown in the following Table 1, where the underlined data represents data collected for a subject in a breath-holding state.

| ID | Cardiac position (distance: m) | | | | | | Torso center (distance: m) | | | |
|---|---|---|---|---|---|---|---|---|---|---|
| 1 | 0.2 | 0.2 | 0.4 | 0.6 | 0.8 | 1 | 0.2 | 0.2 | 0.4 | 0.6 |
| 2 | 0.2 | 0.4 | 0.4 | 0.6 | 0.8 | 1 | 0.2 | 0.4 | 0.4 | 0.6 |
| 3 | 0.2 | 0.4 | 0.6 | 0.6 | 0.8 | 1 | 0.2 | 0.4 | 0.6 | 0.6 |
| 4 | 0.2 | 0.4 | 0.6 | 0.8 | 0.8 | 1 | 0.2 | 0.2 | 0.4 | 0.6 |
| 5 | 0.2 | 0.4 | 0.6 | 0.8 | 1 | 1 | 0.2 | 0.4 | 0.6 | 0.6 |

The following Table 2 shows an accuracy evaluation result of the obtained cardiac motion features by using single-band filtering and multi-band filtering methods respectively to perform signal processing on the collected data via the above method, wherein, the Inter-Beat Interval (IBI) error being less than 100 ms is taken as a standard for evaluation.

TABLE 2
Methods

| | Single-band filtering | | Multi-band filtering | |
|---|---|---|---|---|
| | (5~21 Hz) | | (5~13 Hz, 9~17 Hz, 13~21 Hz) | |
| | | Signals with top | | Signals with top ten |
| | All | ten signal quality | | signal quality |
| | signals | evaluation values | All signals | evaluation values |
| Accuracy | 73.41% | 75.62% | 78.02% | 79.75% |

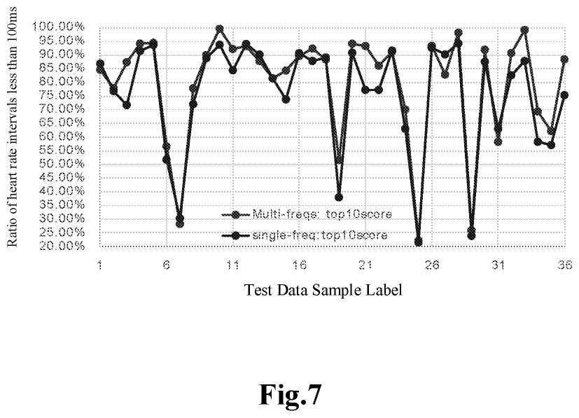

FIG. 7 is a curve schematic diagram of said evaluation result. As shown in Table 2 and FIG. 7, compared with a method for determining a cardiac motion feature by using all signals and only performing single-band filtering, a method for screening a signal by using multi-band filtering and according to signal quality evaluation values can significantly improve accuracy of an obtained cardiac motion feature.

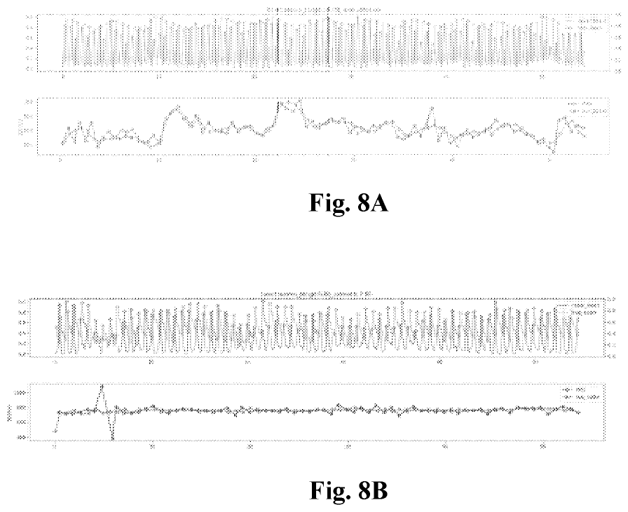

FIG. 8A and FIG. 8B are curve graphs of an experiment result of a method for determining a cardiac motion feature in the embodiments of the present disclosure. FIG. 8A shows a data collection result when a subject is in a sitting position. FIG. 8B shows a data collection result when a subject is in a standing position. Upper parts in FIG. 8A and FIG. 8B are curve graphs of cardiac motion signals obtained according to a method for determining a cardiac motion feature in the embodiments of the present disclosure. Lower parts in FIG. 8A and FIG. 8B are curve graphs of a cardiac motion feature obtained according to a method for determining a cardiac motion feature in the embodiments of the present disclosure, wherein, the cardiac motion feature takes Inter-Beat Interval (IBI) as an example. As shown in FIG. 8A and FIG. 8B, through the above method for determining a cardiac motion feature in the embodiments of the present disclosure, regardless of whether a human body is in a standing or sitting position, cardiac motion signals of the human body can be obtained via collected wireless signals in a case where good accuracy is ensured, and a cardiac motion feature is determined according to the cardiac motion signals. Therefore, the method for determining a cardiac motion feature provided by the embodiments of the present disclosure has no strict requirements on a posture of the human body, and can accurately measure cardiac motions of the human body when the human body is in various postures, and has better applicability.

Through the method for determining a cardiac motion feature provided by the embodiments of the present disclosure, in a non-contact manner, a user has no need to wear any wearable device, and since frequency bands in a heartbeat frequency range are used to filter collected signals, there are no restrictions on a setting location of the device, it is easy to implement and has a wide range of applicability, and has higher privacy protectiveness for the user. In addition, a multi-band filter may further be used to filter the collected signals, so that it is applicable to a wider group of people. Operations such as signal quality evaluation, screening, and energy homogenization are performed based on the filtered signal, which may remove abnormal signals and noise, so it has a strong anti-noise capability, and a resulting cardiac motion signal has higher precision, thereby accuracy of a cardiac motion feature can be ensured.

Each of the above embodiments is only exemplary description of the method in the embodiments of the present disclosure, but the present disclosure is not limited to this, appropriate modifications may be further made based on the above each embodiment. For example, each of the above embodiments may be used individually, or one or more of the above embodiments may be combined.

According to the embodiments of the present disclosure, a cardiac motion feature is determined in a non-contact manner, by performing processing such as filtering, quality evaluation and selection on obtained wireless signals, accuracy of the obtained cardiac motion feature is ensured, and it is also easy to implement, simple to operate, and has a strong anti-noise capability and high privacy protectiveness.

### Embodiments of a Second Aspect

Embodiments of the present disclosure provide an apparatus for determining a cardiac motion feature. The principle of the apparatus to solve the problem is similar to the method in the embodiments of the first aspect, thus its specific implementation can refer to the implementation of the method in the embodiments of the first aspect, the same contents will not be repeated.

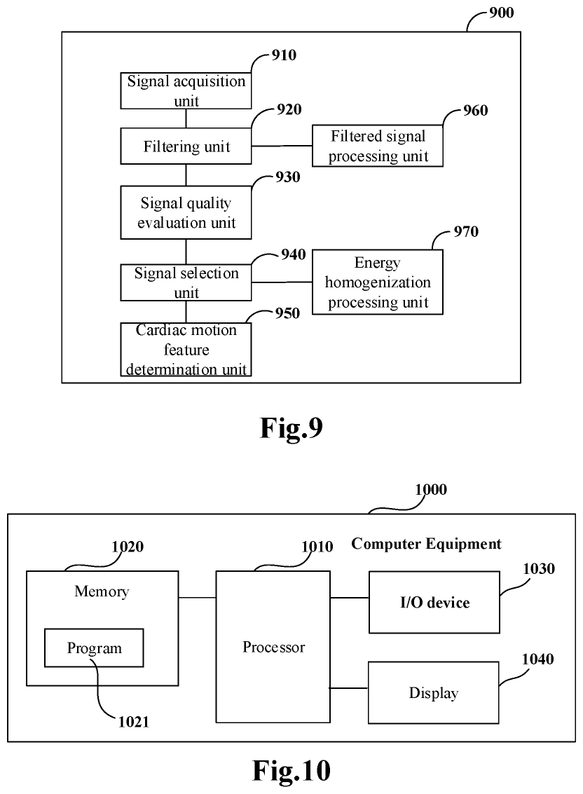

FIG. 9 is a schematic diagram of an apparatus for determining a cardiac motion feature in the embodiments of the present disclosure. As shown in FIG. 9, an apparatus 900 for determining a cardiac motion feature in the embodiments of the present disclosure comprises:
a signal acquisition unit 910, configured to acquire wireless signals reflected by multiple body parts via a wireless sensing device;
a filtering unit 920, configured to filter the acquired wireless signals using a single-band filter or a multi-band filter, to obtain filtered time domain signals;
a signal quality evaluation unit 930, configured to calculate signal quality evaluation values for evaluating signal quality of the filtered time domain signals;
a signal selection unit 940, configured to select candidate signals from the filtered time domain signals according to the signal quality evaluation values; and
a cardiac motion feature determination unit 950, configured to determine a cardiac motion feature based on the candidate signals.

In some embodiments, the apparatus 900 for determining a cardiac motion feature further comprises:
a filtered signal processing unit 960, configured to calculate signal energy values of the filtered time domain signals in a fixed time window; and
in the above embodiment, the signal quality evaluation unit 930 calculates the signal quality evaluation values based on the signal energy values.

In some embodiments, in each fixed time window, a signal energy value at each moment of the filtered time domain signals is equal to a sum of signal energy values at a current moment and preceding moments.

In some embodiments, a length of the fixed time window is a pre-set value, and the filtered time domain signals in the fixed time window includes one heartbeat cycle and is less than one respiratory cycle.

In some embodiments, the apparatus 900 for determining a cardiac motion feature further comprises:
an energy homogenization processing unit 970, configured to perform energy homogenization processing on the candidate signals to obtain cardiac motion signals; and
in the above embodiment, the cardiac motion feature determination unit 950 determines the cardiac motion feature based on the cardiac motion signals.

In some embodiments, the energy homogenization processing unit 970 performs energy homogenization processing on the candidate signals to obtain candidate signals which have been subjected to energy homogenization processing; and performs weighted summing or weighted averaging on the candidate signals which have been subjected to energy homogenization processing, to obtain the cardiac motion signals.

In some embodiments, the candidate signals which have been subjected to energy homogenization processing is represented as follows:

$${s}_{e\_k \_equal} = [ {s}_{e\_k \_ t 1 \_ equal} , {s}_{e\_k \_t 2 \_ equal} , … , {s}_{e\_k \_ t \_ equal} ] ;$$

where, each candidate signal which has been subjected to energy homogenization processing is a sequence of a time duration t1, t2, . . . , t, and s_e_k_equal represents k^{th} candidate signal which has been subjected to homogenization processing.

In the above embodiment, the signal quality evaluation unit 930 performs a fast Fourier transform on signal energy values, i.e., time domain energy values, of the filtered time domain signals in the fixed time window, to obtain frequency domain energy values of frequency domain signals, and divides the frequency domain signals into low band signals and heartbeat band signals according to the frequency domain energy values of the frequency domain signals; and determines the signal quality evaluation values according to the low band signals and the heartbeat band signals.

In the above embodiment, when there is and only one extreme value point in a frequency domain energy value sequence of the heartbeat band, the signal quality evaluation unit 930 calculates a ratio of a frequency domain energy value at the extreme value point to a maximum frequency domain energy value of the low band signals, and takes the ratio as the signal quality evaluation value; when the number of extreme value points in the frequency domain energy value sequence of the heartbeat band is greater than 1, the signal quality evaluation unit 930 calculates a maximum frequency domain energy value in all the extreme value points, then calculates a ratio of the maximum frequency domain energy value to the maximum frequency domain energy value in the low band signals, and takes the ratio as the signal quality evaluation value; and when there is no extreme value point in the frequency domain energy value sequence of the heartbeat band, the signal quality evaluation unit 930 calculates a minimum frequency domain energy value in the heartbeat band signals, then calculates a ratio of the minimum frequency domain energy value to the maximum frequency domain energy value in the low band signals, and takes the ratio as the signal quality evaluation value.

In the above embodiment, the signal selection unit 940 sorts the signal quality evaluation values and selects n filtered signals with higher signal quality evaluation values as the candidate signals, where n is a natural number; or, the signal selection unit 940 calculates a signal quality evaluation threshold based on a maximum value in the signal quality evaluation values, and selects filtered signals with signal quality evaluation values greater than the signal quality evaluation threshold as the candidate signals.

For the sake of simplicity, FIG. 9 only exemplarily shows a connection relationship or signal direction between components or modules, however persons skilled in the art should know that various relevant technologies such as bus connection may be used. The above components or modules can be realized by a hardware facility such as a processor, a memory, etc. The embodiments of the present disclosure have no limitation to this.

Each of the above embodiments is only illustrative for the embodiments of the present disclosure, but the present disclosure is not limited to this, appropriate modifications may be further made based on the above each embodiment. For example, each of the above embodiments may be used individually, or one or more of the above embodiments may be combined.

According to the embodiments of the present disclosure, a cardiac motion feature is determined in a non-contact manner, by performing processing such as filtering, quality evaluation and selection on obtained wireless signals, accuracy of the obtained cardiac motion feature is ensured, and it is also easy to implement, simple to operate, and has a strong anti-noise capability and high privacy protectiveness.

### Embodiments of a Third Aspect

Embodiments of the present disclosure provide a computer equipment, comprising the apparatus 900 for determining a cardiac motion feature as described in the embodiments of the second aspect, whose contents are incorporated here. The computer equipment may be, for example, a computer, server, a workstation, a laptop computer, a smartphone, etc.; however, the embodiments of the present disclosure are not limited to this.

FIG. 10 is a schematic diagram of a computer equipment in the embodiments of the present disclosure. As shown in FIG. 10, the computer equipment 1000 may comprise: a processor (such as a central processing unit (CPU)) 1010 and a memory 1020; the memory 1020 is coupled to the central processor 1010. The memory 1020 may store various data; moreover, further stores a program 1021 for information processing, and executes the program 1021 under the control of the processor 1010.

In some embodiments, the function of the apparatus 900 for determining a cardiac motion feature is integrated into the processor 1010 for implementation. The processor 1010 is configured to implement a method for determining a cardiac motion feature as described in the embodiments of the first aspect.

In some embodiments, the apparatus 900 for determining a cardiac motion feature is configured separately from the processor 1010, for example the apparatus 900 for determining a cardiac motion feature is configured as a chip connected to the processor 1010, a function of the apparatus 900 for determining a cardiac motion feature is realized through the control of the processor 1010.

In addition, as shown in FIG. 10, the computer equipment 1000 may further comprise: an input/output (I/O) device 1030 and a display 1040, etc; wherein the functions of said components are similar to relevant arts, and are not repeated here. It's worth noting that the computer equipment 1000 does not have to include all the components shown in FIG. 10. Moreover, the computer equipment 1000 may also include components not shown in FIG. 10, relevant technologies may be referred to.

Embodiments of the present disclosure further provide a computer readable program, wherein when an apparatus for determining a cardiac motion feature executes the program, the program enables the apparatus for determining a cardiac motion feature to perform the method described in the embodiments of the first aspect.

Embodiments of the present disclosure further provide a storage medium in which a computer readable program is stored, wherein the computer readable program enables an apparatus for determining a cardiac motion feature to execute the method as described in the embodiments of the first aspect.

The apparatus and method in the present disclosure may be realized by hardware, or may be realized by combining hardware with software. The present disclosure relates to such a computer readable program, when the program is executed by a logic component, the computer readable program enables the logic component to realize the device described in the above text or a constituent component, or enables the logic component to realize various methods or steps described in the above text. The present disclosure further relates to a storage medium storing the program, such as a hard disk, a magnetic disk, an optical disk, a DVD, a flash memory and the like.

By combining with the method/device described in the embodiments of the present disclosure, it may be directly reflected as hardware, a software executed by a processor, or a combination of the two. For example, one or more in the functional block diagram or one or more combinations in the functional block diagram as shown in the drawings may correspond to software modules of a computer program flow, and may also correspond to hardware modules. These software modules may respectively correspond to the steps as shown in the drawings. These hardware modules may be realized by solidifying these software modules e.g. using a field-programmable gate array (FPGA).

A software module may be located in a RAM memory, a flash memory, a ROM memory, an EPROM memory, an EEPROM memory, a register, a hard disk, a mobile magnetic disk, a CD-ROM or a storage medium in any other form as known in this field. A storage medium may be coupled to a processor, thereby enabling the processor to read information from the storage medium, and to write the information into the storage medium; or the storage medium may be a constituent part of the processor. The processor and the storage medium may be located in an ASIC. The software module may be stored in a memory of a mobile terminal, and may also be stored in a memory card of the mobile terminal. For example, if a device (such as the mobile terminal) adopts a MEGA-SIM card with a larger capacity or a flash memory apparatus with a large capacity, the software module may be stored in the MEGA-SIM card or the flash memory apparatus with a large capacity.

One or more in the functional block diagram or one or more combinations in the functional block diagram as described in the drawings may be implemented as a general-purpose processor for performing the functions described in the present disclosure, a digital signal processor (DSP), an application-specific integrated circuit (ASIC), a field-programmable gate array (FPGA) or other programmable logic device, discrete gate or transistor logic device, discrete hardware components or any combination thereof. One or more in the functional block diagram or one or more combinations in the functional block diagram as described in the drawings may further be implemented as a combination of computer equipments, such as a combination of a DSP and a microprocessor, a plurality of microprocessors, one or more microprocessors combined and communicating with the DSP or any other such configuration.

The present disclosure is described by combining with the specific implementations, however persons skilled in the art should clearly know that these descriptions are exemplary and do not limit the protection scope of the present disclosure. Persons skilled in the art may make various variations and modifications to the present disclosure according to the spirit and principle of the present disclosure, these variations and modifications are also within the scope of the present disclosure.

Regarding the above implementations disclosed in this embodiment, the following supplements are further disclosed:
1. A method for determining a cardiac motion feature, wherein the method includes:
acquiring wireless signals reflected by multiple body parts via a wireless sensing device;
filtering the acquired wireless signals using a single-band filter or a multi-band filter, to obtain filtered time domain signals;
calculating signal quality evaluation values for evaluating signal quality of the filtered time domain signals;
selecting candidate signals from the filtered time domain signals according to the signal quality evaluation values; and
determining a cardiac motion feature based on the candidate signals.
2. The method according to Supplement 1, wherein calculating the signal quality evaluation values includes:
calculating signal energy values of the filtered time domain signals in a fixed time window; and
determining the signal quality evaluation values based on the signal energy values.
3. The method according to Supplement 2, wherein in each fixed time window, a signal energy value at each moment of the filtered time domain signals is equal to a sum of signal energy values at a current moment and preceding moments.
4. The method according to Supplement 3, wherein a length of the fixed time window is a pre-set value, and the filtered time domain signals in the fixed time window comprises one heartbeat cycle and is less than one respiratory cycle.
5. The method according to Supplement 1, wherein determining the cardiac motion feature includes:
performing energy homogenization processing on the candidate signals to obtain cardiac motion signals; and
determining the cardiac motion feature based on the cardiac motion signals.
6. The method according to Supplement 5, wherein performing energy homogenization processing on the candidate signals includes:
performing energy homogenization processing on the candidate signals to obtain candidate signals which have been subjected to energy homogenization processing; and
performing weighted summing or weighted averaging on the candidate signals which have been subjected to energy homogenization processing, to obtain the cardiac motion signals.
7. The method according to Supplement 6, wherein the candidate signals which have been subjected to energy homogenization processing is represented as follows:

$${s}_{e\_k \_equal} = [ {s}_{e\_k \_ t 1 \_ equal} , {s}_{e\_k \_ t 2 \_ equal} , … , {s}_{e\_k \_ t \_ equal} ] ;$$

where, each candidate signal which has been subjected to energy homogenization processing is a sequence of a time duration t1, t2, . . . , t, and s_e_k_equal represents k^{th} candidate signal which has been subjected to homogenization processing.
8. The method according to Supplement 2, wherein determining the signal quality evaluation values includes:
performing a fast Fourier transform on signal energy values, i.e., a time domain energy values, of the filtered time domain signals in the fixed time window, to obtain frequency domain energy values of frequency domain signals, and dividing the frequency domain signals into low band signals and heartbeat band signals according to the frequency domain energy values of the frequency domain signals; and
determining the signal quality evaluation values according to the low band signals and the heartbeat band signals.
9. The method according to Supplement 8, wherein
when there is and only one extreme value point in a frequency domain energy value sequence of the heartbeat band, a ratio of a frequency domain energy value at the extreme value point to a maximum frequency domain energy value of the low band signals is calculated, and the ratio is taken as a signal quality evaluation value;
when the number of extreme value points in the frequency domain energy value sequence of the heartbeat band is greater than 1, a maximum frequency domain energy value in all the extreme value points is calculated, then a ratio of the maximum frequency domain energy value to the maximum frequency domain energy value in the low band signals is calculated, and the ratio is taken as a signal quality evaluation value; and
when there is no extreme value point in the frequency domain energy value sequence of the heartbeat band, a minimum frequency domain energy value in the heartbeat band signals is calculated, then a ratio of the minimum frequency domain energy value to the maximum frequency domain energy value in the low band signals is calculated, and the ratio is taken as a signal quality evaluation value.
10. The method according to Supplement 1, wherein selecting candidate signals includes:
sorting the signal quality evaluation values and selecting n filtered signals with higher signal quality evaluation values as the candidate signals, where n is a natural number; or,
calculating a signal quality evaluation threshold based on a maximum value in the signal quality evaluation values, and selecting filtered signals with signal quality evaluation values greater than the signal quality evaluation threshold as the candidate signals.

1. An apparatus to determine a cardiac motion feature, the apparatus comprising:
a memory; and
a processor coupled to the memory to:
acquire wireless signals reflected by multiple body parts via a wireless sensing device;
filter the acquired wireless signals, using a single-band filter or a multi-band filter, to obtain filtered time domain signals;
calculate signal quality evaluation values to evaluate signal quality of the filtered time domain signals;
select candidate signals from the filtered time domain signals according to the signal quality evaluation values; and
determine a cardiac motion feature based on the candidate signals.

2. The apparatus according to claim 1, wherein the processor of the apparatus is further configured to:
calculate signal energy values of the filtered time domain signals in a fixed time window; and
calculate the signal quality evaluation values based on the signal energy values.

3. The apparatus according to claim 2, wherein
in each fixed time window, a signal energy value at each moment of the filtered time domain signals is equal to a sum of signal energy values at a current moment and preceding moments.

4. The apparatus according to claim 3, wherein
a length of the fixed time window is a pre-set value, and the filtered time domain signals in the fixed time window comprises one heartbeat cycle and is less than one respiratory cycle.

5. The apparatus according to claim 1, wherein the processor of the apparatus is further configured to:
perform an energy homogenization processing on the candidate signals to obtain cardiac motion signals; and
determine the cardiac motion feature based on the cardiac motion signals.

6. The apparatus according to claim 5, wherein
the energy homogenization processing is performed on the candidate signals to obtain candidate signals which have been subjected to the energy homogenization processing; and
the processor of the apparatus is further configured to perform weighted summing or weighted averaging on the candidate signals which have been subjected to the energy homogenization processing, to obtain the cardiac motion signals.

7. The apparatus according to claim 6, wherein the candidate signals which have been subjected to the energy homogenization processing is represented as follows:
$${s}_{e\_k \_equal} = [ {s}_{e\_k \_ t 1 \_ equal} , {s}_{e\_k \_ t 2 \_ equal} , … , {s}_{e\_k \_ t \_ equal} ]$$
where, each candidate signal which has been subjected to the energy homogenization processing is represented by a sequence of a time duration t1, t2, . . . , t, and S_e_k_equal represents k^{th} candidate signal which has been subjected to the energy homogenization processing.

8. The apparatus according to claim 2, wherein the processor of the apparatus is further configured to:
perform a fast Fourier transform on signal energy values, including at least time domain energy values of the filtered time domain signals in the fixed time window, to obtain frequency domain energy values of frequency domain signals, and divides the frequency domain signals into low band signals and heartbeat band signals according to the frequency domain energy values of the frequency domain signals; and
determines the signal quality evaluation values according to the low band signals and the heartbeat band signals.

9. The apparatus according to claim 8, wherein
based on there being only one extreme value point in a frequency domain energy value sequence of the heartbeat band signals, the processor calculates a ratio of a frequency domain energy value at an extreme value point to a maximum frequency domain energy value of low band signals, and takes the ratio as a signal quality evaluation value;
based on there being a number of extreme value points in the frequency domain energy value sequence of the heartbeat band signals is greater than 1, the processor calculates a maximum frequency domain energy value in all the extreme value points, calculates a ratio of the maximum frequency domain energy value to the maximum frequency domain energy value in the low band signals, and takes the ratio as the signal quality evaluation value; and
based on there being no extreme value point in the frequency domain energy value sequence of the heartbeat band signals, the processor calculates a minimum frequency domain energy value in the heartbeat band signals, and calculates a ratio of the minimum frequency domain energy value to the maximum frequency domain energy value in the low band signals, and takes the ratio as the signal quality evaluation value.

10. The apparatus according to claim 1, wherein the processor of the apparatus is further configured to:
sort the signal quality evaluation values and select n filtered signals with higher signal quality evaluation values as the candidate signals, where n is a natural number; or,
calculate a signal quality evaluation threshold based on a maximum value in the signal quality evaluation values, and select filtered signals with respective signal quality evaluation values greater than the signal quality evaluation threshold as the candidate signals.
